(19) United States
(12) Patent Application Publication
(10) Pub. No.: US 2025/0249256 A1
(43) Pub. Date: Aug. 7, 2025

(54) TITRATION OF VAGUS NERVE STIMULATION

(71) Applicant: The Alfred E. Mann Foundation for Scientific Research, Valencia, CA (US)

(72) Inventors: Brian SHELTON, Ventura, CA (US); Neil Hamilton TALBOT, La Crescenta, CA (US); Hitesh HOTLANI, Santa Clarita, CA (US)

(21) Appl. No.: 19/044,427

(22) Filed: Feb. 3, 2025

Related U.S. Application Data
(60) Provisional application No. 63/549,358, filed on Feb. 2, 2024.

(51) Int. Cl. A61N 1/36; A61N 1/05; A61N 1/375

(52) U.S. Cl. CPC A61N 1/36139; A61N 1/05; A61N 1/36053; A61N 1/36114; A61N 1/3615; A61N 1/36185; A61N 1/375

(57) ABSTRACT

The present disclosure provides systems and methods for vagus nerve stimulation based on adaptive stimulation algorithms.

### CROSS-REFERENCE TO RELATED APPLICATION

The present application claims the benefit of priority to U.S. Provisional Application No. 63/549,358, filed Feb. 2, 2024, the entire contents of which is incorporated by reference in its entirety.

### TECHNICAL FIELD

The present disclosure relates to systems for vagus nerve stimulation and related methods that implement adaptive stimulation algorithms.

### BACKGROUND

Vagus nerve stimulation (VNS) may cause undesirable side effects in addition to the anticipated therapeutic effect. These side effects may include laryngeal effects such as voice changes, sore throat, bradycardia and tachycardia. Bradycardia and tachycardia are of particular concern as they can lead to serious complications. In current VNS systems, the stimulation amplitude is titrated slowly up from a minimum pulse amplitude with increases happening on the order of every week or every other week. The result of this overly conservative titration is that it can take 6 months or more to see a clinical effect.

As stimulation amplitude is increased, type A fibers in the vagus nerve will be recruited first. These include the motor neurons for the larynx. As stimulation amplitude increases, type B fibers will be recruited. These include the afferent fibers that cause the intended therapeutic effect for certain maladies, e.g. epilepsy and depression, and efferent fibers that can cause undesirable side effects, e.g. tachycardia and bradycardia. Thus, limiting the stimulation below the level of any heart-rate effects may be the limit the overall effectiveness and/or delay the onset of benefit of the therapy.

### SUMMARY OF SELECTED ASPECTS

The devices, systems, and methods for VNS described herein address various shortcomings in the art, e.g., by utilizing algorithms and/or stimulation parameters based on sensor data collected from the subject in order to mitigate the issue of tachycardia and bradycardia associated with previous VNS-based therapeutic methods. In some aspects, titration of VNS is performed at levels higher than typical for prior methods by increasing the stimulation pulse amplitude either to or near the neural fulcrum. As explained herein (and illustrated by FIG. 3), as VNS is increased, there will be no heart rate change observed up to a certain amplitude. Above this amplitude, the heart rate will increase to a point and then begin to drop. The neural fulcrum is defined as that stimulation amplitude where the heart rate has dropped back to its original rate. In some aspects, the best electrode or electrodes for administering stimulation may be selected by minimizing laryngeal muscle activation, and/or cardiac/heart rate changes Titration may be initiated, e.g., at a pulse amplitude below the amplitude that causes laryngeal muscle contraction. As described in further detail herein, the high level of stimulation of the vagus nerve provided by the present systems and methods allows for effective treatment of multiple conditions while reducing, if not eliminating, the problems of tachycardia and bradycardia

In a first general aspect, the disclosure provides a system for VNS, comprising: a stimulator implanted in a subject and configured to deliver electrical stimulation to a vagus nerve of the subject via one or more electrodes; and a controller configured to control delivery of the electrical stimulation to the vagus nerve of the subject, by titrating a pulse amplitude of stimulation delivered via at least one of the one or more electrodes, based on a predetermined electromyography (EMG) activation threshold for the at least one electrode, and a predetermined neural fulcrum stimulation amplitude for the at least one electrode.

In some aspects, the controller is further configured to control delivery of the electrical stimulation to the vagus nerve of the subject, by titrating the pulse amplitude of stimulation delivered via at least one of the one or more electrodes, within a range defined by a predetermined EMG activation threshold for the at least one electrode, and a predetermined neural fulcrum stimulation amplitude for the at least one electrode.

In some aspects, the one or more electrodes comprises a plurality of electrodes, and the controller is further configured to control delivery of the electrical stimulation to the vagus nerve of the subject, by titrating a pulse amplitude of stimulation delivered via each of the plurality of electrodes, from a predetermined EMG activation threshold for each respective electrode to the predetermined neural fulcrum stimulation amplitude for each respective electrode.

In some aspects, the predetermined EMG activation threshold for each electrode comprises a minimum pulse amplitude of stimulation previously determined to evoke an EMG response in the subject.

In some aspects, the predetermined neural fulcrum stimulation amplitude for each electrode is higher than the respective EMG activation threshold for the electrode, and previously determined to evoke no change in the subject's heart rate during stimulation.

In some aspects, the one or more electrodes comprises a plurality of electrodes, and the controller is further configured to select the electrode(s) that has/have either the highest EMG activation threshold(s), or to avoid use of the electrodes with the lowest EMG activation threshold(s), and to cause delivery of the electrical stimulation to the vagus nerve of the subject, by titrating the pulse amplitude of stimulation delivered via the selected electrode(s). As noted herein, the highest or lowest electrode(s) may comprise a single highest or lowest electrode, or a plurality (e.g., the two highest, three highest, four highest, or two lowest, three lowest, four lowest, etc.). In cases where a plurality of electrodes are selected for use (or avoidance), any number of electrodes may be selected (e.g., 1, 2, 3, 4, 5, 6, 7, 8, 9, or 10 electrodes).

In some aspects, the system further comprises. one or more sensors, each configured to detect or measure a signal indicative of a biomarker of the subject; wherein the controller is further configured to determine a heart rate of the subject based on data received from the one or more sensors, and to control delivery of the electrical stimulation to the vagus nerve of the subject by titrating a pulse amplitude of stimulation delivered via at least one of the one or more electrodes across a range defined by a first amplitude and a second amplitude, wherein (i) the first amplitude is the predetermined EMG activation threshold for the at least one of the one or more electrodes, and (ii) the second amplitude is an amplitude equal to or greater than a predetermined neural fulcrum stimulation amplitude for the at least one of the one or more electrodes, and below a predetermined threshold.

In some aspects, the controller is further configured to titrate the pulse amplitude of stimulation delivered via at least one of the one or more electrodes downward in response to determining that the heart rate of the subject: (a) has increased following stimulation, when the pulse amplitude of stimulation delivered via at least one of the one or more electrodes is above the predetermined neural fulcrum stimulation amplitude for the at least one of the one or more electrodes, or (b) has exceeded a predetermined threshold. For example, in some aspects, the controller may be configured to titrate the pulse amplitude over a period of one or more days or weeks (e.g., over at least 1, 2, 3, 4, 5, 6, 7, or 8 days or weeks The titration may be based on logged heart rate or any other biometric data described herein, e.g., logged in a memory accessible to the controller or an electronic device configured to communicate with the controller.

In some aspects, the controller is configured to titrate the pulse amplitude of stimulation delivered via at least one of the one or more electrodes, based on the heart rate of the subject, in order to maintain stimulation at an amplitude at or within a predetermined threshold (e.g., within 1, 2, 3, 4, 5, 6, 7, 8, 9, 10, 11, 12, 13, 14, 15, 16, 17, 18, 19, or 20%, or within 0.1, 0.2, 0.3, 0.4 or 0.5 mA) from the amplitude associated with the neural fulcrum for the subject.

In some aspects, the one or more sensors comprises at least one sensor configured to detect or measure a signal indicative of a position and/or activity level of the subject; and the controller is further configured to titrate the pulse amplitude of stimulation delivered via at least one of the one or more electrodes upward, or downward, based on the position and/or activity level of the subject.

In some aspects, the one or more sensors comprise: a) one or more implanted sensors, optionally comprising one or more sensors at least partially contained in a housing of the simulator; b) one or more external sensors; or c) a combination of implanted and external sensors, optionally comprising one or more sensors at least partially contained in a housing of the simulator.

In a second general aspect, the disclosure provides methods for providing VNS using any of the systems described herein. Such methods may comprise, e.g., administering stimulation to a vagus nerve of a subject, using an implanted stimulator configured to deliver electrical stimulation to the vagus nerve of the subject via one or more electrodes; wherein a pulse amplitude of the administered stimulation is set by a controller communicatively linked to the implanted stimulator, the controller being configured to titrate a pulse amplitude of stimulation delivered via at least one of the one or more electrodes, based on a predetermined EMG activation threshold for the at least one electrode, and a predetermined neural fulcrum stimulation amplitude for the at least one electrode.

In some aspects of the methods described herein, the controller is further configured to titrate the pulse amplitude of stimulation delivered via at least one of the one or more electrodes, within a range defined by a predetermined EMG activation threshold for the at least one electrode, and a predetermined neural fulcrum stimulation amplitude for the at least one electrode.

In some aspects of the methods described herein, the one or more electrodes comprises a plurality of electrodes, and the controller is further configured to titrate a pulse amplitude of stimulation delivered via each of the plurality of electrodes, from a predetermined EMG activation threshold for each respective electrode to the predetermined neural fulcrum stimulation amplitude for each respective electrode.

In some aspects of the methods described herein, the predetermined EMG activation threshold for each electrode comprises a minimum pulse amplitude of stimulation previously determined to evoke an EMG response in the subject.

In some aspects of the methods described herein, the predetermined neural fulcrum stimulation amplitude for each electrode is higher than the respective EMG activation threshold for the electrode, and previously determined to evoke no change in the subject's heart rate during stimulation.

In some aspects of the methods described herein, the one or more electrodes comprises a plurality of electrodes, and the controller is further configured to: select the electrode that has either the lowest EMG activation threshold or the lowest neural fulcrum stimulation amplitude; and to cause delivery of the electrical stimulation to the vagus nerve of the subject, by titrating the pulse amplitude of stimulation delivered via the selected electrode.

In some aspects, the methods described herein further comprise the steps of: receiving, by the controller, sensor data indicative of a detection or measurement of a biomarker of the subject; and determining, by the controller, a heart rate of the subject based on the received sensor data, wherein the controller is further configured to titrate the pulse amplitude of stimulation delivered via at least one of the one or more electrodes across a range defined by a first amplitude and a second amplitude, wherein (i) the first amplitude is the predetermined EMG activation threshold for the at least one of the one or more electrodes, and (ii) the second amplitude is an amplitude greater than a predetermined neural fulcrum stimulation amplitude for the at least one of the one or more electrodes, and below a predetermined threshold.

In some aspects of the methods described herein, the controller is further configured to titrate the pulse amplitude of stimulation delivered via at least one of the one or more electrodes downward in response to determining that the heart rate of the subject: (a) has increased following stimulation, when the pulse amplitude of stimulation delivered via at least one of the one or more electrodes is above the predetermined neural fulcrum stimulation amplitude for the at least one of the one or more electrodes, or (b) has exceeded a predetermined threshold.

In some aspects, the methods described herein further comprise the steps of: receiving, by the controller, sensor data indicative of a detection or measurement of a position and/or activity level of the subject; wherein the controller is further configured to titrate the pulse amplitude of stimulation delivered via at least one of the one or more electrodes downward based on the position and/or activity level of the subject.

In some aspects of the methods described herein, the sensor data indicative of (i) a detection or measurement of a biomarker of the subject, and/or (ii) a position and/or activity level of the subject, is received from: a) one or more implanted sensors, optionally comprising one or more sensors at least partially contained in a housing of the simulator; b) one or more external sensors; or c) a combination of implanted and external sensors, optionally comprising one or more sensors at least partially contained in a housing of the simulator.

### BRIEF DESCRIPTION OF THE DRAWINGS

FIG. 1 is a block diagram illustrating an exemplary embodiment of a system for titrating VNS in accordance with the present disclosure.

FIG. 2 is a conceptual flow diagram of a process for fitting a subject using one of the adaptive VNS systems described herein.

FIG. 3 is a graph showing the change in heart rate (bpm) as pulse amplitude (mA) is increased, with the neural fulcrum annotated.

FIG. 4 is a conceptual flow diagram of a process for treating a subject using one of the adaptive VNS systems described herein.

FIG. 5 is a is a block diagram of various example system components, capable of being used along the lines as described in example implementations in accordance with aspects of the present disclosure.

The exemplary systems and methods shown in the foregoing drawings represent non-limiting examples and do not constrain the scope of the inventions described herein, which are defined solely by the claims.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

Several aspects of exemplary embodiments according to the present disclosure will now be presented with reference to various systems and methods. These systems and methods will be described in the following detailed description and illustrated in the accompanying drawings by various blocks, components, circuits, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system.

By way of example, an element, or any portion of an element, or any combination of elements may be implemented as a “processing system” or “controller” that includes one or more processors. Examples of processors include microprocessors, microcontrollers, graphics processing units (“GPUs”), central processing units (“CPUs”), application processors, digital signal processors (“DSPs”), reduced instruction set computing (“RISC”) processors, systems on a chip (“SoC”), baseband processors, field programmable gate arrays (“FPGAs”), programmable logic devices (“PLDs”), application-specific integrated circuits (“ASICs”), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in one or more exemplary embodiments, the functions described may be implemented in hardware, software, or any combination thereof. If implemented in software, the functions may be stored on or encoded as one or more instructions or code on a computer-readable medium. Computer-readable media includes computer storage media. Storage media may be any available media that can be accessed by a computer. By way of example, and not limitation, such computer-readable media can comprise a random-access memory (“RAM”), a read-only memory (“ROM”), an electrically erasable programmable ROM (“EEPROM”), optical disk storage, magnetic disk storage, other magnetic storage devices, combinations of the aforementioned types of computer-readable media, or any other medium that can be used to store computer executable code in the form of instructions or data structures that can be accessed by a computer.

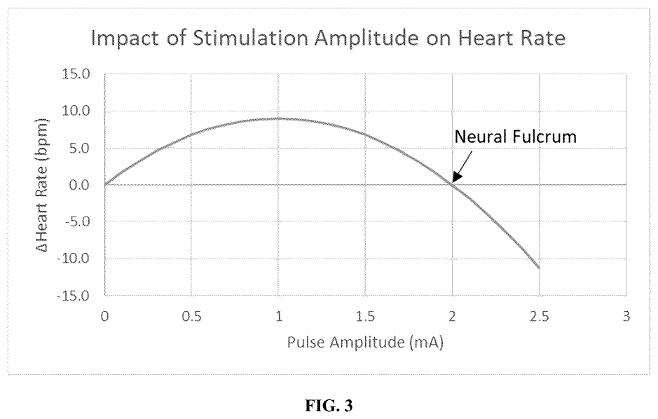

The present disclosure is based in part on findings that have revealed new insights into the heart rate side effect that patients undergoing VNS can suffer. As the level of VNS is increased above a certain threshold (typically different for each patient), the patient may experience an increase in heart rate (tachycardia). However, as the level of stimulation increases further, the heart rate typically levels out and then starts to decline. The decline in heart rate will quickly drop to the neural fulcrum (see FIG. 3) where there is no heart rate response to stimulation. Bradycardia will typically accelerate and be exacerbated as stimulation continues to increase, potentially becoming unhealthy, or even life threatening. Both tachycardia and bradycardia are undesirable conditions if they are more than a few beats per minute. However, it is desirable to maximize the stimulation of the vagus nerve in order to maximize the beneficial effects such as reduction in depression, epileptic seizure frequency, inflammation related maladies such as rheumatoid arthritis, sympathetic tone related issues such as excessive nervousness, auto-immune conditions, and others.

The present disclosure provides systems and methods of titrating VNS intensity either to or beyond the neural fulcrum “hump,” where the heart rate is increased above normal, to the point where the rate is on a downward trend and equal or close to the patient's normal heart rate. As described herein, a level of stimulation that elicits a normal heart rate response may is achievable using a closed-loop control system with heart rate measurement as an input. The heart rate may be measured by one or more of several methods such as electrocardiogram (ECG) measurement, inertial measurements, photoplethysmography, and others, during fitting sessions. The subject's heart rate may also be measured using one or more implanted or external sensors (e.g., a sensor integrated into the implanted stimulator).

In some aspects, the systems described herein may further be configured to monitor and/or account for other factors, such as the subject's activity level. Activity level may be determined, e.g., using one or more sensors integrated into or communicatively linked with an implanted stimulator. External sensors may also be used, such as an inertial measurement unit (IMU), in order to detect and/or measure chest or diaphragm expansion or motion (by the subject generally, or of one or more anatomical locations). The closed-loop control algorithms described herein may also include safety thresholds for a minimum and maximum heart rate where the device may be programmed to cease stimulation once those limits are crossed.

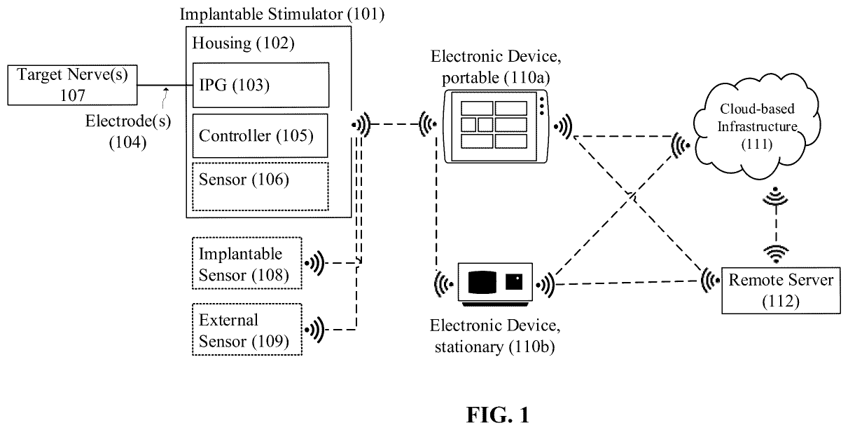

FIG. 1 is a block diagram of an exemplary adaptive VNS system according to the present disclosure. This particular example illustrates a system in the form of an implantable stimulator (101) structured as a housing (102) comprising an IPG (103), electrodes (104) for stimulating at least one target nerve (107) of the subject, and an integrated sensor (106). In this case, the integrated sensor (106) is shown to be optional. An additional implantable sensor (108) and external sensor (109) are also shown as optional components. The implantable stimulator (101) is shown to be capable of wireless communication with the optional implantable sensor (108) and external sensor (109), as well as with a portable electronic device (110a). The portable electronic device may comprise, e.g., a dedicated controller, or a smartphone, or other portable electronic device configured to execute one or more software applications or executable code that allows the device to interact with and/or control one or more parameters of the controller (105) of the implantable stimulator (101). A stationary electronic device (110b) is also shown as another potential component of the system. Stationary devices are envisioned as devices that are typically operated while stationary (e.g., a desktop computer), and which are configured to execute one or more software applications or executable code configured to interact with and/or control one or more parameters of the controller (105) of the implantable stimulator (101). In this case, the controller (105) is shown to wirelessly communicate with the portable electronic device (110a). However, in other aspects it may communicate with a stationary electronic device (110b) or with any of the other potential components of systems described herein. In some aspects, the controller (105) may communicate with a portable electronic device (110a) or a stationary electronic device (110b) that is in turn configured to communicate with one or more external sensors (109). For example, the controller (105) may be capable of obtaining sensor data from a user's smartwatch (or any other device comprising one or more sensors capable of detecting a signal indicative of a biometric parameter of the subject), or from a portable or stationary electronic device (110a, 110b) that has received sensor data from a paired or otherwise communicatively linked device. FIG. 1 further illustrates the potential use of a remote server (112) and cloud-based infrastructure (111). Remote servers (112) may be used, e.g., to store logs comprising sensor and/or stimulation-related data (e.g., to allow a medical practitioner or clinician programmer to review such data). In some aspects, a remote server (112) may be configured to set or modify one or more parameters of the controller (105) of the implantable stimulator (101). For example, a remote medical practitioner or clinician programmer may be allowed to review sensor and/or stimulation-related data for the subject (e.g., stored on the remote server 112) and to adjust one or more thresholds or other parameters related to treatment. In some aspects, the remote server (112) may also serve as a repository for sensor data collected by third-party devices (e.g., sensors in a user's smartphone or smartwatch, etc.), that is made accessible to the controller (105) or to any communicatively-linked devices such as the portable or stationary electronic devices (110a, 110b) shown in this example. Such data may be used by the systems described herein, e.g., to set parameters for treatment.

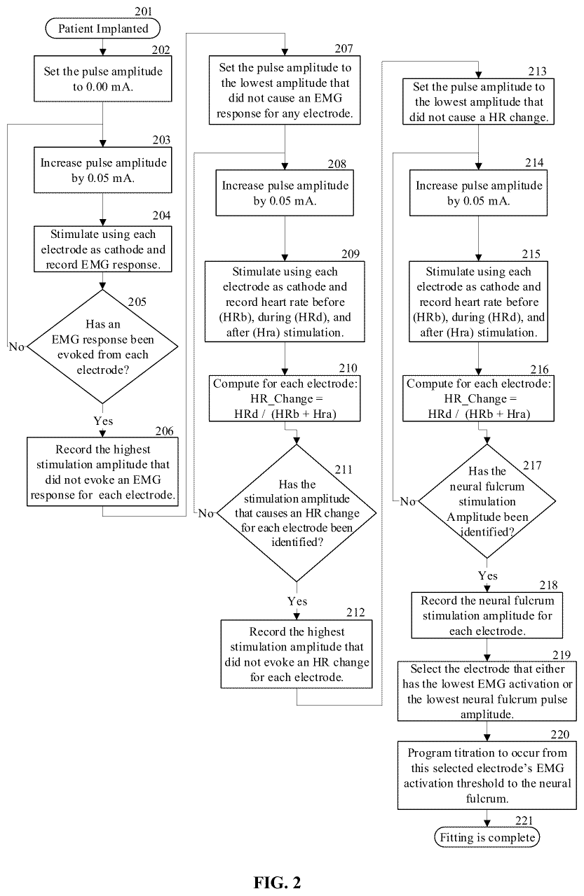

FIG. 2 is flowchart showing an exemplary method for fitting a subject with an adaptive VNS system according to the present disclosure. In this example, the fitting process begins with the implantation of an implantable stimulator (step 201). A clinician programmer may set the initial pulse amplitude of stimulation to a low level (e.g., 0 mA, as shown here (step 202). Stimulation may then be increased by a predetermined or other amount (e.g., 0.05 mA) (step 203). Stimulation may then be provided using electrode of the implanted system as a cathode (step 204), and an electromyogram (EMG) response is evaluated (step 205). If no response is detected, the pulse amplitude would increase further (i.e., returning to step 203). If a response is detected, the fitting process proceeds to step 206, where the highest stimulation amplitude that did not evoke an EMG response is recorded for each of the electrodes.

Next, the system may be configured to set the pulse amplitude of stimulation to the lowest amplitude that did not cause an EMG response for any electrode (step 207). Again, pulse amplitude stimulation is increased (here, by 0.05 mA) (step 208), and stimulation is provided, with the subject's heart rate being measured before (“HRb”), during (“HRd”) and after (“HRa”) stimulation (step 209). A change in heart rate (“HR_Change”) is computed for each electrode (step 210). As shown here, if the stimulation amplitude that causes a heart rate change for each electrode is identified (Step 211), fitting may proceed to step (212), if not, the process returns to step 208. In this example, the highest amplitude that did not evoke a heart rate change is recorded for each electrode (step 212).

Next, the process continues by a clinician programmer setting the pulse amplitude to the lowest amplitude that did not cause a heart rate change (step 213), and once again titrating the pulse amplitude of stimulation upwards (here, by 0.05 mA) (step 214). Stimulation is provided using each electrode as cathode and the subject's HRb, HRd, and HRa levels are measured (step 215), and an HR_Change parameter is again computed for each electrode (step 216).

The change in heart rate is used to determine whether the neural fulcrum has been reached (step 217). As explained above, and shown in FIG. 3, as the pulse amplitude of stimulation is increased, subjects will typically experience tachycardia, with the change in heart rate gradually decreasing and plateauing at the neural fulcrum. Further increases in pulse amplitude will then normally result in a decrease in the change in heart rate, followed by bradycardia as the subject's heart rate level continues to decrease. In the exemplary fitting process shown in FIG. 2, the pulse amplitude associated with the neural fulcrum for the subject may be recorded (step 218), and stimulation parameters may then be set for the subject taking this result into account. For example, the electrode that has the lowest EMG activation or the lowest neural fulcrum pulse amplitude may be selected (step 219), and the implanted stimulator (101) may be configured to apply stimulation at a pulse amplitude between the selected electrode's EMG activation threshold and the neural fulcrum pulse amplitude. Here, the system is configured to titrate a pulse amplitude between these two thresholds (step 220).

In some aspects, the system may be configured to increase or titrate the pulse amplitude of stimulation, between the selected electrode's EMG activation threshold and the neural fulcrum pulse amplitude, based on an evaluation of the subject's heart rate or a change in the subject's heart rate. For example, the present systems may be configured to monitor the subject's heart rate using any of the sensors described herein, and to titrate the pulse amplitude upwards when a positive change in heart rate is detected, with the pulse amplitude associated with neural fulcrum operating as an upper endpoint. In some aspects, the system may be configured to decrease the pulse amplitude when a negative change in heart rate is detected at a pulse amplitude level below the pulse amplitude associated with the neural fulcrum. In some aspects, the system is configured to apply a pulse amplitude configured to maintain a subject's heart rate within a predetermined range. For example, the system may be configured to pause stimulation, or decrease the pulse amplitude of stimulation, when the subject's heart rate has increased by 1, 2, 3, 4, 5, 6, 7, 8, 9, or 10 bpm compared to a predetermined value or a baseline value established measured prior to the initiation of stimulation. In other aspects, stimulation may be paused, or the pulse amplitude of stimulation may be decreased, when the subject's heart rate increases by 1, 2, 3, 4, 5, 6, 7, 8, 9 or 10% compared to a predetermined value or a baseline value established measured prior to the initiation of stimulation. In alternative aspects, stimulation may be paused, or the pulse amplitude of stimulation may be decreased, when the subject's heart rate increases by an amount or percentage within a range defined by any pair of endpoints selected from either of the foregoing lists).

The system shown in FIG. 2 is exemplary and non-limiting with respect to the process steps and sequence, as well as the parameters (e.g., thresholds) used in this embodiment. For example, alternative embodiments of this system are envisioned wherein the pulse amplitude of stimulation is adjusted (e.g., at steps 203, 208, and/or 214) by different amounts (e.g., by 0.01, 0.02, 0.03, 0.04, 0.05, 0.06, m 0.07, 0.08, 0.09, 0.10, 0.11, 0.12, 0.13, 0.14, 0.15, 0.016, 0.17, 0.18, 0.19, 0.20, 0.21, 0.22, 0.23, 0.24, 0.25, 0.30, 0.35, 0.40, 0.45, or 0.50 mA, or by an amount within a range defined by a pair of endpoints selected from any of the foregoing amounts. The pulse amplitude increase applied at each of the aforementioned steps may be the same or independently selected. In some aspects, the increase in pulse amplitude may be constant, whereas in others it may increase at a variable rate (e.g., the rate of increase in pulse amplitude may be progressively lowered, allowing for more precise identification as to the threshold that evokes an EMG response). Similarly, the initial pulse amplitude is variable in alternative embodiments. Here, a starting value of 0.00 mA was selected, but in other cases a non-zero starting level may be applied.

In some aspects, stimulus-evoked EMG activity may be evaluated within a window (e.g., 1.0, 1.5, 2.0, 2.5, 3.0, 3.5, 4.0, 4.5, 5.0, 5.5, 60.0, 6.5, 7.0, 7.5, 8.0, 8.5, 9.0, 9.5 10.0, 20.0, 30.0, 40.0, 50.0. 60.0, 70.0, 80.0, 90.0, 100.0, 120.0, 140.0, 160.0, 180.0, 200.0, 220.0, 240.0, 260.0, 280.0, or 300.0 ms, or a window comprising an amount of time within a range defined by any pair of the foregoing lengths of time). In some aspects, an EMG response is determined to occur when stimulation of an electrode results in an EMG voltage above a predetermined threshold (e.g., measured in mV). In some aspects, an EMG response is determined to occur when stimulation of an electrode results in an EMG voltage exceeding a pre-activation baseline level by a predetermined percentage (e.g., by at least 10, 20, 30, 40, or 50%).

In some aspects, the subject's heart rate may be detected using an inertial, electrical, electromagnetic, ultrasound, or optical sensor. For example, a photoplethysmography (PPG) sensor may be located on a wearable device so that the PPG sensor is in contact with a subject's skin. The PPG sensor may detect blood flow beneath the subject's skin and this information may be used to determine the subject's heart rate. In another aspect, the heart rate sensor may be an electromagnetic sensor (e.g., located on a chest strap). In some aspects, an IMU, a phonocardiography (PCG) sensor, or any other sensor capable of detecting a signal that is directly or indirectly indicative of a subject's heart rate. The subject's heart rate may be determined using a single obtained using a single sensor or a plurality of sensors. One or more of the sensors may be integrated into or communicatively linked with the implantable stimulator (101), implanted in the subject (e.g., as an implantable sensor 108), or external to the subject (e.g., external sensor 109). Any external sensors may be integrated into or communicatively linked to the portable or stationary electronic device (110a, 110b). In some aspects, one or more external sensors (109) may be integrated into or communicatively linked to a wearable device (e.g., a smart watch or a wearable fitness monitoring device). The subject's heart rate may be measured by the sensor itself (e.g., using software or firmware executable by a processor integrated into the sensor), by a controller (105) of the implantable stimulator (101), or by any other component of the systems described herein (e.g., by the portable or stationary electronic device (110a, 110b).

In the example shown in FIG. 2, the system is configured to apply stimulation at a pulse amplitude between the selected electrode's EMG activation threshold and the neural fulcrum pulse amplitude. However, in other aspects, the electrode's EMG activation threshold and/or the neural fulcrum pulse amplitude for one or more electrodes may be used to set or control treatment parameters without functioning as endpoints. For example, an alternative system may be configured to apply stimulation using a pulse amplitude above that of the amplitude associated with the neural fulcrum. In that case, sensor data may be collected to monitor the subject's heart rate (e.g., to ensure that stimulation does not cause the subject's heart rate to decrease below a predetermined safety threshold). In some aspects, the system may be configured to decrease the pulse amplitude when a negative change in heart rate is detected at a pulse amplitude level above the pulse amplitude associated with the neural fulcrum. In some aspects, the system is configured to pause stimulation (or to decrease the pulse amplitude of stimulation) when the subject's heart rate has decreased by 1, 2, 3, 4, 5, 6, 7, 8, 9, or 10 bpm compared to a predetermined value or a baseline value established measured prior to the initiation of stimulation. In other aspects, stimulation may be paused, or the pulse amplitude of stimulation may be decreased, when the subject's heart rate decreases by 1, 2, 3, 4, 5, 6, 7, 8, 9 or 10% compared to a predetermined value or a baseline value established measured prior to the initiation of stimulation. In alternative aspects, stimulation may be paused, or the pulse amplitude of stimulation may be decreased, when the subject's heart rate decreases by an amount or percentage within a range defined by any pair of endpoints selected from either of the foregoing lists).

In some aspects, the system may be configured to apply stimulation at a pulse amplitude within 0.1, 0.2, 0.3, 0.4, 0.5, 0.6, 0.7, 0.8, 0.9, or 1.0 mA of a pulse amplitude associated with the neural fulcrum. In some aspects, the system may alternatively be configured to apply stimulation at a pulse amplitude within a range that deviates from the pulse amplitude associated with the neural fulcrum by up to 10, 20, 30, 40, or 50%. For example, the graph shown in FIG. 3 illustrates a situation where the neural fulcrum is associated with a pulse amplitude of 1 mA; a 50% deviation in this hypothetical case would encompass a range of 0.5 to 1.5 mA.

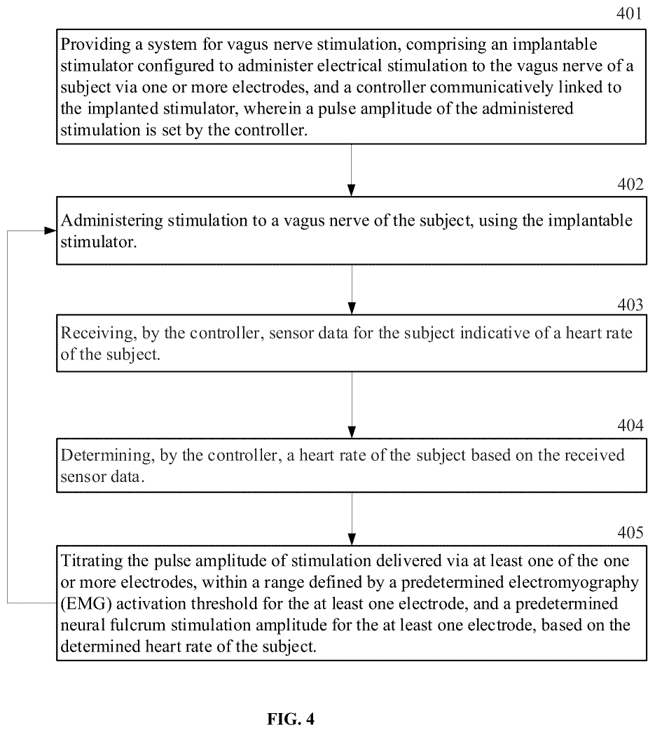

FIG. 4 is a flowchart showing an exemplary method for treating a subject using the present systems. In this non-limiting example, treatment begins with the subject being provided with a system for VNS, comprising an implantable stimulator configured to administer electrical stimulation to the vagus nerve of the subject via one or more electrodes, and a controller communicatively linked to the implanted stimulator, wherein a pulse amplitude of the administered stimulation is set by the controller (step 401). Next, the vagus nerve of the subject is stimulated using the implantable stimulator (step 402). As shown here, the controller may receive sensor data indicative of a heart rate of the subject (step 403) and use this sensor data to determine the subject's heart rate (step 404). The pulse amplitude of stimulation delivered via at least one of the one or more electrodes may then be titrated, by the controller, within a range defined by a predetermined EMG activation threshold for the at least one electrode, and a predetermined neural fulcrum stimulation amplitude for the at least one electrode, based on the determined heart rate of the subject (Step 405). In other aspects, additional sensor data (e.g., movement data collected using an IMU may also be collected and used by the controller as a parameter for determining the level of stimulation to apply during this titration process).

In some aspects, the VNS systems and methods described herein may be used to treat depression, epilepsy (e.g., epileptic seizure frequency), and/or inflammation (e.g., related to maladies such as rheumatoid arthritis), brain diseases (e.g., Alzheimer's disease, dementia, traumatic brain injury, Parkinson's disease, and ischemic stroke), heart conditions (e.g., heart failure, cardiovascular disease, and for improving heart function, pain-related conditions (e.g., Migraines, cluster headaches, and pain-related disorders), inflammatory conditions (e.g., inflammatory bowel disease, rheumatoid arthritis, and autoimmune diseases, psychiatric conditions (e.g., anxiety disorders, and PTSD), and other conditions (e.g., diabetes, obesity, and sleep disorders). An exemplary method of treating any such diseases or conditions may comprise fitting a subject with a system according to the present disclosure and/or using any of the present systems to reduce one or more symptoms of the foregoing diseases and conditions.

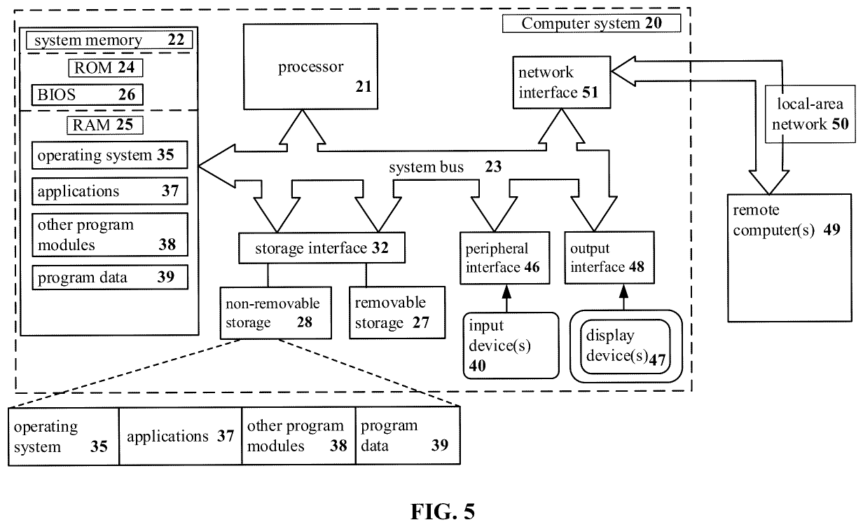

Aspects of the present disclosure may be implemented using hardware, software, or a combination thereof and may be implemented in one or more computer systems or other processing systems. In an aspect of the present disclosure, features are directed toward one or more computer systems capable of carrying out the functionality described herein. FIG. 5 is a block diagram illustrating an example of a computer system 20 which may be used to implement aspects of the systems and methods described herein. The computer system 20 can be in the form of multiple computing devices, or in the form of a single computing device, for example, a desktop computer, a notebook computer, a laptop computer, a mobile computing device, a smart phone, a tablet computer, a server, a mainframe, an embedded device, and other forms of computing devices.

As shown, the computer system 20 includes a central processing unit (CPU) 21, a system memory 22, and a system bus 23 connecting the various system components, including the memory associated with the central processing unit 21. The system bus 23 may comprise a bus memory or bus memory controller, a peripheral bus, and a local bus that is able to interact with any other bus architecture. Examples of the buses may include PCI, ISA, PCI-Express, HyperTransport™, InfiniBand™, Serial ATA, I2C, and other suitable interconnects. The central processing unit 21 (also referred to as a processor) can include a single or multiple sets of processors having single or multiple cores. The processor 21 may execute one or more computer-executable code implementing the techniques of the present disclosure. For example, any of commands/steps discussed in this specification, or shown in the accompanying drawings, may be performed by processor 21. The system memory 22 may be any memory for storing data used herein and/or computer programs that are executable by the processor 21. The system memory 22 may include volatile memory such as a random access memory (RAM) 25 and non-volatile memory such as a read only memory (ROM) 24, flash memory, etc., or any combination thereof. The basic input/output system (BIOS) 26 may store the basic procedures for transfer of information between elements of the computer system 20, such as those at the time of loading the operating system with the use of the ROM 24.

The computer system 20 may include one or more storage devices such as one or more removable storage devices 27, one or more non-removable storage devices 28, or a combination thereof. The one or more removable storage devices 27 and non-removable storage devices 28 are connected to the system bus 23 via a storage interface 32. In an aspect, the storage devices and the corresponding computer-readable storage media are power-independent modules for the storage of computer instructions, data structures, program modules, and other data of the computer system 20. The system memory 22, removable storage devices 27, and non-removable storage devices 28 may use a variety of computer-readable storage media. Examples of computer-readable storage media include machine memory such as cache, SRAM, DRAM, zero capacitor RAM, twin transistor RAM, eDRAM, EDO RAM, DDR RAM, EEPROM, NRAM, RRAM, SONOS, PRAM; flash memory or other memory technology such as in solid state drives (SSDs) or flash drives; magnetic cassettes, magnetic tape, and magnetic disk storage such as in hard disk drives or floppy disks; optical storage such as in compact disks (CD-ROM) or digital versatile disks (DVDs); and any other medium which may be used to store the desired data and which can be accessed by the computer system 20.

The system memory 22, removable storage devices 27, and non-removable storage devices 28 of the computer system 20 may be used to store an operating system 35, additional program applications 37, other program modules 38, and program data 39. The computer system 20 may include a peripheral interface 46 for communicating data from input devices 40, such as a keyboard, mouse, stylus, game controller, voice input device, touch input device, or other peripheral devices, such as a printer or scanner via one or more I/O ports, such as a serial port, a parallel port, a universal serial bus (USB), or other peripheral interface. A display device 47 such as one or more monitors, projectors, or integrated display, may also be connected to the system bus 23 across an output interface 48, such as a video adapter. In addition to the display devices 47, the computer system 20 may be equipped with other peripheral output devices (not shown), such as loudspeakers and other audiovisual devices.

The computer system 20 may operate in a network environment, using a network connection to one or more remote computers 49. The remote computer (or computers) 49 may be local computer workstations or servers comprising most or all of the aforementioned elements in describing the nature of a computer system 20. Other devices may also be present in the computer network, such as, but not limited to, routers, network stations, peer devices or other network nodes. The computer system 20 may include one or more network interfaces 51 or network adapters for communicating with the remote computers 49 via one or more networks such as a local-area computer network (LAN) 50, a wide-area computer network (WAN), an intranet, and the Internet. Examples of the network interface 51 may include an Ethernet interface, a Frame Relay interface, SONET interface, and wireless interfaces.

Aspects of the present disclosure may be a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store program code in the form of instructions or data structures that can be accessed by a processor of a computing device, such as the computing system 20. The computer readable storage medium may be an electronic storage device, a magnetic storage device, an optical storage device, an electromagnetic storage device, a semiconductor storage device, or any suitable combination thereof. By way of example, such computer-readable storage medium can comprise a random access memory (RAM), a read-only memory (ROM), EEPROM, a portable compact disc read-only memory (CD-ROM), a digital versatile disk (DVD), flash memory, a hard disk, a portable computer diskette, a memory stick, a floppy disk, or even a mechanically encoded device such as punch-cards or raised structures in a groove having instructions recorded thereon. As used herein, a computer readable storage medium is not to be construed as being transitory signals per se, such as radio waves or other freely propagating electromagnetic waves, electromagnetic waves propagating through a waveguide or transmission media, or electrical signals transmitted through a wire.

Computer readable program instructions described herein can be downloaded to respective computing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network interface in each computing device receives computer readable program instructions from the network and forwards the computer readable program instructions for storage in a computer readable storage medium within the respective computing device.

Computer readable program instructions for carrying out operations of the present disclosure may be assembly instructions, instruction-set-architecture (ISA) instructions, machine instructions, machine dependent instructions, microcode, firmware instructions, state-setting data, or either source code or object code written in any combination of one or more programming languages, including an object-oriented programming language, and conventional procedural programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a LAN or WAN, or the connection may be made to an external computer (for example, through the Internet). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

In various aspects, the systems and methods described in the present disclosure can be addressed in terms of modules. The term “module” as used herein refers to a real-world device, component, or arrangement of components implemented using hardware, such as by an application specific integrated circuit (ASIC) or FPGA, for example, or as a combination of hardware and software, such as by a microprocessor system and a set of instructions to implement the module's functionality, which (while being executed) transform the microprocessor system into a special-purpose device. A module may also be implemented as a combination of the two, with certain functions facilitated by hardware alone, and other functions facilitated by a combination of hardware and software. In certain implementations, at least a portion, and in some cases, all, of a module may be executed on the processor of a computer system. Accordingly, each module may be realized in a variety of suitable configurations, and should not be limited to any particular implementation exemplified herein.

In the interest of clarity, not all of the routine features of the aspects are disclosed herein. It would be appreciated that in the development of any actual implementation of the present disclosure, numerous implementation-specific decisions must be made in order to achieve the developer's specific goals, and these specific goals will vary for different implementations and different developers. It is understood that such a development effort might be complex and time-consuming, but would nevertheless be a routine undertaking of engineering for those of ordinary skill in the art, having the benefit of this disclosure.

Furthermore, it is to be understood that the phraseology or terminology used herein is for the purpose of description and not of restriction, such that the terminology or phraseology of the present specification is to be interpreted by the skilled in the art in light of the teachings and guidance presented herein, in combination with the knowledge of those skilled in the relevant art(s). Moreover, it is not intended for any term in the specification or claims to be ascribed an uncommon or special meaning unless explicitly set forth as such.

The various aspects disclosed herein encompass present and future known equivalents to the known modules referred to herein by way of illustration. Moreover, while aspects and applications have been shown and described, it would be apparent to those skilled in the art having the benefit of this disclosure that many more modifications than mentioned above are possible without departing from the inventive concepts disclosed herein.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain aspects of the present disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the present disclosure to be practiced otherwise than specifically described herein. Accordingly, this disclosure includes all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing aspects of the present disclosure (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or “exemplary” language (e.g., “such as”) provided herein is intended merely to better illuminate the present disclosure and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present disclosure. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only, and is not intended to limit the scope of the present invention, which is defined solely by the claims. Accordingly, the present invention is not limited to that precisely as shown and described.

1. A system for vagus nerve stimulation, comprising:
a stimulator implanted in a subject and configured to deliver electrical stimulation to a vagus nerve of the subject via one or more electrodes or electrode pairs; and
a controller configured to
control delivery of the electrical stimulation to the vagus nerve of the subject, by titrating a pulse amplitude of stimulation delivered via at least one of the one or more electrodes, based on (a) a predetermined electromyography (EMG) activation threshold for the at least one electrode, and (b) (i) a predetermined neural fulcrum stimulation amplitude for the at least one electrode, or (ii) a stimulation amplitude found to cause a change in a heart rate of the subject.

2. The system of claim 1, wherein the controller is further configured to
control delivery of the electrical stimulation to the vagus nerve of the subject, by titrating the pulse amplitude of stimulation delivered via at least one of the one or more electrodes, within a range defined by a predetermined electromyography (EMG) activation threshold for the at least one electrode, and a predetermined neural fulcrum stimulation amplitude for the at least one electrode.

3. The system of claim 1, wherein the controller is further configured to
control delivery of the electrical stimulation to the vagus nerve of the subject, by titrating the pulse amplitude of stimulation delivered via at least one of the one or more electrodes, within a range defined by a predetermined electromyography (EMG) activation threshold for the at least one electrode, and a predetermined stimulation amplitude found to cause an increase in heart rate in the subject, for the at least one electrode.

4. The system of claim 1, wherein the one or more electrodes comprises a plurality of electrodes, and the controller is further configured to
control delivery of the electrical stimulation to the vagus nerve of the subject, by titrating a pulse amplitude of stimulation delivered via each of the plurality of electrodes, from a predetermined EMG activation threshold for each respective electrode to the predetermined neural fulcrum stimulation amplitude for each respective electrode.

5. The system of claim 1, wherein the predetermined EMG activation threshold for each electrode comprises a minimum pulse amplitude of stimulation previously determined to evoke an EMG response in the subject.

6. The system of claim 1, wherein the predetermined neural fulcrum stimulation amplitude for each electrode is higher than the respective EMG activation threshold for the electrode, and previously determined to evoke no change in the subject's heart rate during stimulation.

7. The system of claim 1, wherein the one or more electrodes comprises a plurality of electrodes, and the controller is further configured to
select the electrode that has either the lowest EMG activation threshold or the lowest neural fulcrum stimulation amplitude, and to
cause delivery of the electrical stimulation to the vagus nerve of the subject, by titrating the pulse amplitude of stimulation delivered via the selected electrode.

8. The system of claim 1, wherein the system further comprises
one or more sensors, each configured to detect or measure a signal indicative of a biomarker of the subject,
wherein the controller is further configured to determine a heart rate of the subject based on data received from the one or more sensors, and to
control delivery of the electrical stimulation to the vagus nerve of the subject by titrating a pulse amplitude of stimulation delivered via at least one of the one or more electrodes across a range defined by a first amplitude and a second amplitude,
wherein (i) the first amplitude is the predetermined EMG activation threshold for the at least one of the one or more electrodes, and (ii) the second amplitude is an amplitude greater than a predetermined neural fulcrum stimulation amplitude for the at least one of the one or more electrodes, and below a predetermined threshold.

9. The system of claim 8, wherein the controller is further configured to titrate the pulse amplitude of stimulation delivered via at least one of the one or more electrodes downward in response to determining that the heart rate of the subject:
(a) has increased following stimulation, when the pulse amplitude of stimulation delivered via at least one of the one or more electrodes is above the predetermined neural fulcrum stimulation amplitude for the at least one of the one or more electrodes, or
(b) has exceeded a predetermined threshold.

10. The system of claim 9, wherein the one or more sensors comprises at least one sensor configured to detect or measure a signal indicative of a position and/or activity level of the subject; and
the controller is further configured to titrate the pulse amplitude of stimulation delivered via at least one of the one or more electrodes downward based on the position and/or activity level of the subject.

11. The system of claim 8, wherein the one or more sensors comprise:
a) one or more implanted sensors, optionally comprising one or more sensors at least partially contained in a housing of the simulator;
b) one or more external sensors; or
c) a combination of implanted and external sensors, optionally comprising one or more sensors at least partially contained in a housing of the simulator.

12. A method for providing vagus nerve stimulation, comprising:
administering stimulation to a vagus nerve of a subject, using an implanted stimulator configured to deliver electrical stimulation to the vagus nerve of the subject via one or more electrodes;
wherein a pulse amplitude of the administered stimulation is set by a controller communicatively linked to the implanted stimulator, the controller being configured to titrate a pulse amplitude of stimulation delivered via at least one of the one or more electrodes, based on a predetermined electromyography (EMG) activation threshold for the at least one electrode, and a predetermined neural fulcrum stimulation amplitude for the at least one electrode.

13. The method of claim 12, wherein the controller is further configured to
titrate the pulse amplitude of stimulation delivered via at least one of the one or more electrodes, within a range defined by a predetermined electromyography (EMG) activation threshold for the at least one electrode, and a predetermined neural fulcrum stimulation amplitude for the at least one electrode.

14. The method of claim 12, wherein the one or more electrodes comprises a plurality of electrodes, and the controller is further configured to
titrate a pulse amplitude of stimulation delivered via each of the plurality of electrodes, from a predetermined EMG activation threshold for each respective electrode to the predetermined neural fulcrum stimulation amplitude for each respective electrode.

15. The method of claim 12, wherein the predetermined EMG activation threshold for each electrode comprises a minimum pulse amplitude of stimulation previously determined to evoke an EMG response in the subject.

16. The method of claim 12, wherein the predetermined neural fulcrum stimulation amplitude for each electrode is higher than the respective EMG activation threshold for the electrode, and previously determined to evoke no change in the subject's heart rate during stimulation.

17. The method of claim 12, wherein the one or more electrodes comprises a plurality of electrodes, and the controller is further configured to
select the electrode that has either the lowest EMG activation threshold or the lowest neural fulcrum stimulation amplitude; and to
cause delivery of the electrical stimulation to the vagus nerve of the subject, by titrating the pulse amplitude of stimulation delivered via the selected electrode.

18. The method of claim 12, wherein the method further comprises the steps of:
receiving, by the controller, sensor data indicative of a detection or measurement of a biomarker of the subject; and
determining, by the controller, a heart rate of the subject based on the received sensor data;
wherein the controller is further configured to titrate the pulse amplitude of stimulation delivered via at least one of the one or more electrodes across a range defined by a first amplitude and a second amplitude, wherein (i) the first amplitude is the predetermined EMG activation threshold for the at least one of the one or more electrodes, and (ii) the second amplitude is an amplitude greater than a predetermined neural fulcrum stimulation amplitude for the at least one of the one or more electrodes, and below a predetermined threshold.

19. The method of claim 18, wherein the controller is further configured to titrate the pulse amplitude of stimulation delivered via at least one of the one or more electrodes downward in response to determining that the heart rate of the subject:
(a) has increased following stimulation, when the pulse amplitude of stimulation delivered via at least one of the one or more electrodes is above the predetermined neural fulcrum stimulation amplitude for the at least one of the one or more electrodes, or
(b) has exceeded a predetermined threshold.

20. The method of claim 19, further comprising a step of:
receiving, by the controller, sensor data indicative of a detection or measurement of a position and/or activity level of the subject;
wherein the controller is further configured to titrate the pulse amplitude of stimulation delivered via at least one of the one or more electrodes downward based on the position and/or activity level of the subject.

21. The method of claim 18, wherein the sensor data indicative of (i) a detection or measurement of a biomarker of the subject, and/or (ii) a position and/or activity level of the subject, is received from:
a) one or more implanted sensors, optionally comprising one or more sensors at least partially contained in a housing of the stimulator;
b) one or more external sensors; or
c) a combination of implanted and external sensors, optionally comprising one or more sensors at least partially contained in a housing of the simulator.
